(19) United States
(12) Plant Patent Application Publication
(10) Pub. No.: US 2017/0071086 P1
(43) Pub. Date: Mar. 9, 2017

(54) Cladrastis tree named `White Rain'

(50) ;

(71) Applicant: Peter A. Driscoll, Beallsville, MD (US)

(72) Inventor: Peter A. Driscoll, Beallsville, MD (US)

(21) Appl. No.: 14/756,460

(22) Filed: Sep. 5, 2015

(51) Int. Cl. A01H 5/00

(52) U.S. Cl. PLT/216

(57) ABSTRACT

A new and distinct cultivar of Cladrastis tree named ‘White Rain’, characterized by its outwardly weeping tree form; moderately vigorous to vigorous growth habit; and numerous pendulous inflorescences with white-colored flowers.

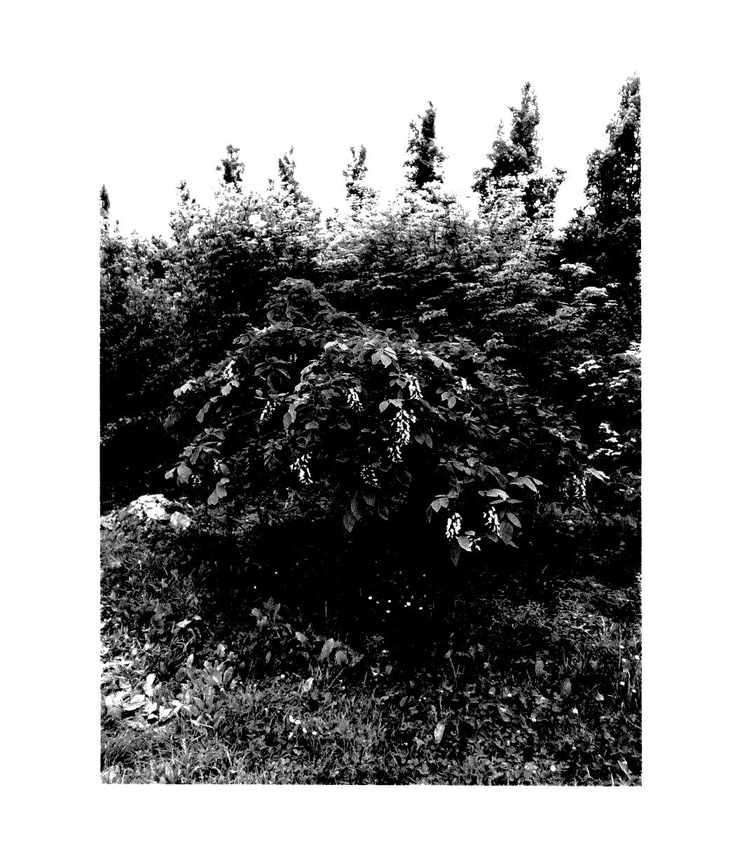

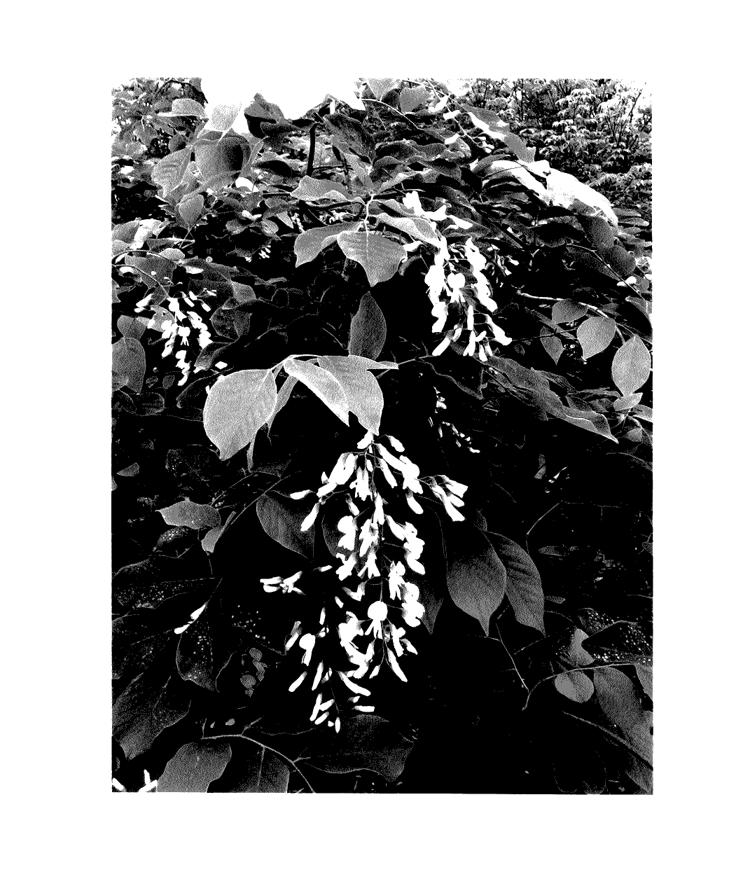

Botanical designation: Cladrastis kentukea.

Cultivar denomination: ‘WHITE RAIN’.

### BACKGROUND OF THE INVENTION

The present invention relates to a new and distinct cultivar of Cladrastis tree, botanically known as Cladrastis kentukea, commercially referred to as Yellowwood and hereinafter referred to by the name ‘White Rain’.

The new Cladrastis tree originated from an open-pollination of an unnamed selection of Cladrastis kentukea, not patented, as the female, or seed, parent with an unknown selection of Cladrastis kentukea as the male, or pollen, parent. The new Cladrastis tree was discovered and selected by the Inventor as a single plant from within the progeny of the stated open-pollination in a controlled environment in Beallsville, Md. during the spring of 2002.

Asexual reproduction of the new Cladrastis tree by side veneer grafting in a controlled greenhouse environment in Decherd, Tenn. since February, 2012 has shown that the unique features of this new Cladrastis tree are stable and reproduced true to type in successive generations.

### SUMMARY OF THE INVENTION

Trees of the new Cladrastis have not been observed under all possible combinations of environmental and cultural conditions. The phenotype may vary somewhat with variations in environmental conditions such as temperature and light intensity without, however, any variance in genotype.

The following traits have been repeatedly observed and are determined to be the unique characteristics of ‘White Rain’. These characteristics in combination distinguish ‘White Rain’ as a new and distinct Cladrastis tree:
1. Outwardly weeping tree form.
2. Moderately vigorous to vigorous growth habit.
3. Numerous pendulous inflorescences with white-colored flowers.

Trees of the new Cladrastis can be compared to trees of the female parent selection. Trees of the new Cladrastis differ primarily from trees of the female parent selection in tree form as trees of the female parent selection are upright and not weeping in form.

### BRIEF DESCRIPTION OF THE PHOTOGRAPHS

The accompanying colored photographs illustrate the overall appearance of the new Cladrastis tree showing the colors as true as it is reasonably possible to obtain in colored reproductions of this type.

Colors in the photographs may differ slightly from the color values cited in the detailed botanical description which accurately describe the colors of the new Cladrastis tree.

The photograph on the first sheet is a side perspective view of a typical flowering 13-year old tree of ‘White Rain’ grown during the spring in an outdoor nursery.

The photograph on the second sheet is a close-up view of a typical flowering tree of ‘White Rain’.

### DETAILED BOTANICAL DESCRIPTION

The aforementioned photographs and following observations, measurements and values describe trees grown in an outdoor nursery in Decherd, Tenn. and under cultural practices typical of commercial Cladrastis tree production. Trees used in the aforementioned photographs and the following description were 13 years old. During the production of the trees, average day temperature was 20° C. and average night temperature was 18° C. In the following description, color references are made to The Royal Horticultural Society Colour Chart, 2007 Edition, except where general terms of ordinary dictionary significance are used.
Botanical classification: Cladrastis kentukea ‘White Rain’.
Parentage:
   Female, or seed, parent.—Unnamed selection of Cladrastis kentukea, not patented.
   Male, or pollen, parent.—Unknown selection of Cladrastis kentukea, not patented.
Propagation:
   Type.—By side veneer grafting or budding.
Tree description:
   Tree form and growth habit.—Deciduous tree with outwardly weeping tree form; freely branching habit with about 15 to 20 lateral branches developing per tree; full and densely-foliated appearance; moderately vigorous to vigorous growth habit.
   Tree height.—About 2.59 meters.
   Tree width (spread).—About 2 meters.
   Trunk caliper.—About [symbol] .
   Growth rate, height.—About 50 cm to 75 cm per year.
   Growth rate, caliper.—About [symbol] .
   Lateral branch angle orientation (angle).— [symbol] .
   Lateral branch length.—About 75 cm to 100 cm.
   Lateral branch diameter.—About 2 cm to 3.5 cm.
   Internode length.—About 6 cm to 9 cm.
   Lateral branch color.—Close to 144A.
   Immature bark texture.—Smooth, glabrous.
   Mature bark texture.—Woody.
   Mature bark color.—Close to [symbol] .
Leaf description:
   Arrangement.—Alternate, odd-pinnately compound with about seven leaflets per leaf.
   Leaf length.—About 15 cm to 20 cm.
   Leaf width.—About 7.5 cm to 11 cm.
   Leaflet length.—About [symbol] .
   Leaflet width.—About [symbol] .
   Leaflet shape.— [symbol] .
   Leaflet apex.— [symbol] .
   Leaflet base.— [symbol] .
   Leaflet margins.—Entire.
   Leaflet venation pattern.—Pinnate.
   Leaflet aspect.—Pendulous.
   Leaflet texture, upper and lower surfaces.—Smooth, glabrous.
   Leaflet luster, upper and lower surfaces.—Matte.
   Leaflet color.—When developing, upper surface: Close to 137A. When developing, lower surface: Close to 144A. Fully developed, upper surface: Close to 136A; venation, close to 137A. Fully developed, lower surface: Close to 141A; venation, close to 145B. Autumn color, upper surface: Close to [symbol] Autumn color, lower surface: Close to [symbol] .
   Petioles.—Length: About 5 cm. Diameter: About 2 mm to 2.5 mm. Texture, upper and lower surfaces: Smooth, glabrous. Color, upper and lower surfaces: Close to 137C.
   Color, lower surface.—Close to 144B.
Flower description:
   Flower arrangement and habit.—Single flowers arranged in pendulous terminal panicled racemes with typically about 20 to 30 flowers developing per inflorescence and about 23 inflorescences developing per tree.
   Fragrance.—Faintly fragrant; pleasant.
   Natural flowering season.—Plants of the new Cladrastis flower during May until early June in Decherd, Tenn.
   Flower longevity.—Individual flowers last about eight to eleven days on the plant; flowers not persistent.
   Inflorescence height.—About 20 cm to 30 cm.
   Inflorescence diameter.—About 10 cm to 15 cm.
   Flower diameter.—About 5 mm to 8 mm.
   Flower length (depth).—About 1 cm to 1.5 cm.
   Flower buds.—Length: About [symbol] Diameter: About [symbol] Texture: Smooth, glabrous. Color: Close to [symbol] .
   Petals.—Quantity: [symbol] Arrangement: [symbol] Length: About [symbol] Width: About [symbol] Shape: [symbol] Apex: [symbol] Margin: [symbol] Texture, upper surface: [symbol] Texture, lower surface: [symbol] Luster, upper surface: [symbol] Luster, lower surface: [symbol] Color: When opening, upper surface: Close to [symbol] When opening, lower surface: Close to [symbol] Fully opened, upper surface: Close to [symbol] Fully opened, lower surface: Close to [symbol]
   Sepals.—Calyx length: About 7.5 mm to 10 mm. Calyx diameter: About 5 mm to 6 mm. Quantity: [symbol] Arrangement: [symbol] Length: About [symbol] Width: About [symbol] Shape: [symbol] Apex: [symbol] Margin: [symbol] Texture, upper surface: [symbol] Texture, lower surface: [symbol] Luster, upper surface: [symbol] Luster, lower surface: [symbol] Color: When opening, upper surface: Close to [symbol] When opening, lower surface: Close to [symbol] Fully opened, upper surface: Close to [symbol] Fully opened, lower surface: Close to [symbol] .
   Peduncles.—Length: About 19 cm to 25 cm. Diameter: About 2 mm. Strength: Strong, flexible. Aspect: [symbol] Texture: [symbol] Color: Close to 138D.
   Pedicels.—Length: About 1 cm to 1.5 cm. Diameter: About 2 mm. Strength: Strong, flexible. Aspect: [symbol] Texture: [symbol] Color: Close to 138D.
   Reproductive organs.—Stamens: Quantity per flower: [symbol] Filament length: About [symbol] Filament color: Close to [symbol] Anther length: About [symbol] Anther diameter: About [symbol] Anther color: Close to [symbol] Pollen amount (abundant, moderate or scarce): [symbol] Pollen color: Close to [symbol] Pistils: Pistil length: About [symbol] Style length: About [symbol] Style texture: [symbol] Style color: Close to [symbol] Stigma shaped: [symbol] Stigma color: Close to [symbol] Ovary color: Close to [symbol] .
   Seeds and fruits.—Seed and fruit development have not been observed on trees of the new Cladrastis.
Temperature tolerance: Trees of the new Cladrastis have been observed to tolerate high temperatures about 35° C., low temperatures about −11° C. and are suitable for USDA Hardiness Zones 4 through 8.
Pathogen & pest resistance: Trees of the new Cladrastis have been not observed to be resistant to pathogens and pests common to Cladrastis trees.

It is claimed:

1. A new and distinct Cladrastis tree named ‘White Rain’ as illustrated and described.
